(19) United States
(12) Patent Application Publication
(10) Pub. No.: US 2010/0004552 A1
(43) Pub. Date: Jan. 7, 2010

(54) METHOD AND DEVICE FOR THE DETERMINATION OF BREATH FREQUENCY

(75) Inventors: Wei Zhang, Niederwerrn (DE); Carsten Mueller, Euerbach (DE)

Correspondence Address: JACOBSON HOLMAN PLLC, 400 SEVENTH STREET N.W., SUITE 600, WASHINGTON, DC, 20004, US

(73) Assignee: FRESENIUS MEDICAL CARE DEUTSCHLAND GMBH, Bad Homburg (DE)

(21) Appl. No.: 12/448,312

(22) PCT Filed: Nov. 28, 2007
(86) PCT No.: PCT/EP2007/010355

§ 371 Date: Jun. 17, 2009

(30) Foreign Application Priority Data
Dec. 21, 2006 (DE) 10 2006 060 819.4

(51) Int. Cl. A61B 5/08

(52) U.S. Cl. 600/529

(57) ABSTRACT

The present invention provides a method for determining the respiratory rate of a patient comprising the steps of determining at least two time dependent respiratory signals by at least two different methods as well as a determining of a respiratory rate on the basis of the at least two time dependent respiratory signals. In this connection, the resulting respective instantaneous respiratory rates fᵢ(n) (i=1, 2, . . . ) are determined from the at least two time dependent respiratory signals sᵢ(t) (i=1, 2, . . . ) and an average respiratory rate f(n) determined by a weighted averaging of the respiratory rates fᵢ(n) (i=1, 2, . . . ) is produced. In this averaging, the weightings kᵢ(n) (i=1, 2, . . . ) of the individual respiratory rates fᵢ(n) (i=1, 2, . . . ) depend on a difference between the respective respiratory rates fᵢ(n) (i=1, 2, . . . ) and an estimate fᵢ(n) which is determined on the basis of at least two respiratory signals sᵢ(t) (i=1, 2, . . . ). An apparatus for carrying out the method is likewise provided.

The present invention relates to a method and to an apparatus for determining the respiratory rate of a patient which serve the metrological monitoring of the respiratory activity of a patient.

A number of different methods are already known in this respect to extract information on the respiratory activity from different physiological measured signals of the patient. It is thus possible to deduce and monitor the respiratory activity of a patient using the following methods:
via the change in the bioimpedance on the basis of the respiratory movement of the thorax, impedance plethysmography (IP);
from the heart rate variability signal, since information on the respiratory activity is contained in the heart rate on the basis of the respiratory sinus arrhythmia;
from an optoelectronic measurement of the blood volume pulse, photoplethysmography (PPG), which contains an additive signal portion based on respiratory induced fluctuations in blood pressure;
from potential differences at the surface of the body based on cardiac activity, the so-called electrocardiogram (ECG);
from the pulse wave transit time of a pulse wave in an artery (PTT) since the fluctuation in the blood pressure comprises a respiratory induced portion and the systolic blood pressure is correlated in almost linear fashion with the pulse wave transit time.

However, the measurements of the respiratory rate based on these methods are influenced by a plurality of interference signals. It has been found in this connection that an evaluation of the quality of the individual signals is practically not possible due to the complexity of the interference signals.

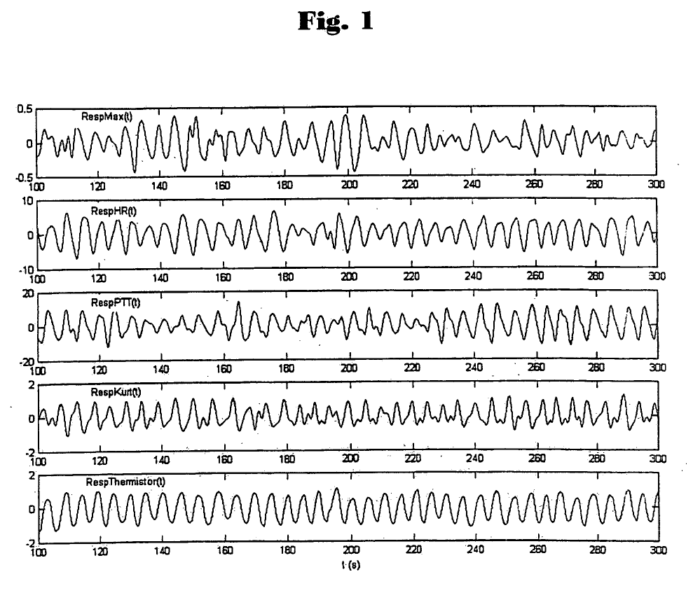

This is basically due to the following three reasons:
Reason 1—Indirect Measurement
   The extraction of the respiratory information, e.g. from the ECG and PPG signals is an indirect measurement of the respiratory activities and thus always prone to interference.
Reason 2—Different Form of the Extracted Respiratory Signals
   It results from the evaluation of the laboratory and clinical data that the form of the respiratory activities in the extracted respiratory signals is dependent on the person and also differs over time, see FIG. 1. It is therefore not possible simply to state that one extracted respiratory signal is definitively better than the others.
Reason 3—Artifacts
   The extracted respiratory signals can be differently affected by artifacts which can result from the different methods or which can e.g. arise due to the movement of the patient or also due to other physiological processes.

To improve the measurement accuracy, it is therefore known from US 2005/0027205 to average a plurality of respiratory rates obtained from different measurement methods. Impedance plethysmography (IP) and photoplethysmography (PPG) are used as methods in this connection and are both dependent on movement. To eliminate the artifacts caused by patient movements, a special mathematical model is used for the determination of prognostic values, said model making a prediction separately foe each of the two measuring channels. The prediction is in each case only based on past measured values for the respective channel as well as on a factor which takes account of conventional deviations in the rate across the board, i.e. the output signal of each channel is smoothed and extrapolated. The measured frequencies are now compared with the forecast values and the weightings for the averaging of the rates are determined via this difference. The weighting in this process is, however, based solely on the difference from the model for the respective measuring channel. This procedure therefore requires that the model describes reality better than the measurements since no feedback takes place from the measured results to the model structure. Interference such as arises due to patient movements can hereby only be attenuated at times, whereas permanent or systematic interference influences cannot be eliminated. However, in particular errors based on physiological interference factors such as Mayer waves are thereby taken over in a displaced manner and further substantially falsify the respiratory rate determined by such a system. The calculation using the prognostic model is moreover expensive and complicated.

It is therefore the object of the present invention to provide an improved method for determining the respiratory rate of a patient which increases the reliability of a specific respiratory rate determined in a simple manner and can in particular also eliminate physiological interference factors.

This object is solved in accordance with the invention by a method for determining the respiratory rate of a patient in accordance with claim 1. Such a method contains the steps of determining at least two time-dependent respiratory signals sᵢ(t) (i=1, 2, . . . ) by at least two different methods and of determining the respective respiratory rates resulting in each case from the at least two time-dependent respiratory signals sᵢ(t) (i=1, 2, . . . ) fᵢ(n) (i=1, 2, . . . ) and the determining of an average respiratory rate f(n) by a weighted averaging of the respiratory rates fᵢ(n) (i=1, 2, . . . ). In this averaging, the weightings kᵢ(n) (i=1, 2, . . . ) of the individual respiratory rates fᵢ(n) (i=1, 2, . . . ) depend on a difference between the respective respiratory rates fᵢ(n) (i=1, 2, . . . ) and an estimate fₛ(n) which is determined on the basis of at least two respiratory signals sᵢ(t) (i=1, 2, . . . ). The weighting therefore no longer takes place separately for each channel, but is based on the difference of the respective respiratory rates fᵢ(n) (i=1, 2, . . . ) from a prognostic value which is determined on the basis of data from a plurality of channels. Since interference usually has a different effect on the different time-dependent respiratory signals sᵢ(t) (i=1, 2, . . . ) and thus also on the respiratory rates determined therefrom fᵢ(n) (i=1,2, . . . ), interference signals and errors in the individual respiratory signals can be suppressed via such a weighting. If interference is only present in one respiratory signal and so in only one respiratory rate, the difference between this respiratory rate and the estimate fₛ will be large, which in turn results in a low weighting of this respiratory rate in the averaging. A feedback on the weighting of the individual channels hereby results by which systematic or permanent interference influences can also be eliminated. It is in particular possible in this way to eliminate the influence of physiological interference factors such as Mayer waves.

The estimate fₛ(n) is advantageously determined on the basis of a preceding mean respiratory rate f(n−1) already determined. The weightings can in particular thus advantageously be determined from the difference between the current respiratory rates fᵢ(n) (i=1, 2, . . . ) measured via the respective channels and the average f(n−1) determined last. If this difference is large, a small weighting is associated with the respective channel, and vice versa. Differences in the individual values are thus always related to the total system so that systematic errors can also be eliminated via this feedback of the system structure of the total system. Further advantageously, the estimate fₛ(n) is determined, in particular for initialization by a combination of rate information from at least two time dependent respiratory signals sᵢ(t) (i=1, 2, . . . ) or by forming an average of the current respiratory rates fᵢ(n) (i=1, 2, . . . ). Since in particular no reliable estimates from previous measurements are present from the start, either averages (normally unweighted) of the current measured values can be used or an estimate can be provided by a combination of rate information. The use of rate information is relatively intensive from a calculation aspect, but does also deliver more precise results. This is in particular of advantage for initialization, but can also be used when no values can be determined in another way due to strong interference.

The at least two time dependent respiratory signals sᵢ(t) (i=1, 2, . . . ) are advantageously determined from measured physiological signals. In this process, the time dependent respiratory signals sᵢ(t) (i=1, 2, . . . ) can be determined by different methods from one or more measured physiological signals, which increases the reliability of the final result.

In this connection, the measured physiological signals advantageously form a selection from the following signals:
bioimpedance signal;
heart rate variability signal;
photoplethysmographic signal (PPG signal);
statistical source signal of the ECG;
pulse wave transit time signal (PTT signal).

A plurality of different physiological signals thus result which can be measured and used to determine the time dependent respiratory signals sᵢ(t) (i=1, 2, . . . ).

In this connection, the determination of the at least two time dependent respiratory signals sᵢ(t) (i=1,2, . . . ) takes place from the measured physiological signals through a band pass filter. Since the physiological signals usually do not only contain information on the respiratory activity, but also other information, e.g. on the heart rate, this information which is not wanted can be filtered by a band pass filter so that the time dependent respiratory signals sᵢ(t) (i=1, 2, . . . ) result from the physiological signals.

The band pass filter advantageously allows frequencies to pass in a range from approx. 0.12 Hz to 0.42 Hz, while other frequencies disposed outside this range of respiratory rates are suppressed.

Advantageously, the method in accordance with the invention furthermore includes the step of determining an instantaneous respiratory rate fᵢ(k) from the time-dependent respiratory signal sᵢ(t) (i=1, 2, . . . ) by determining the time index tₘₐₓ(k) of the maxima of the time dependent respiratory signal sᵢ(t) (i=1, 2, . . . ). The respiratory rate can thus be determined in a simple manner from the time dependent respiratory signal sᵢ(t) (i=1, 2, . . . ) by determining its maximum or by determining the time indices of the maxima. In this connection, the determination of the instantaneous respiratory rate advantageously takes place by the determination of the time interval tₘₐₓ(k)-tₘₐₓ(k−1) between adjacent maxima of the time dependent respiratory signal.

The time interval between two successive maxima of the time dependent respiratory signal is inversely proportional to the instantaneous respiratory rate fᵢ(k). The instantaneous respiratory rate is advantageously determined from three respiratory signals: fₕᵣ(m), fₐₘₚ(n), fₚₜₜ(k). A consistency check of the time indices m, n and k advantageously takes place. The time indices have to be within a predetermined time window for this purpose. 50% of the current respiratory period can, for example, be used for the time window.

The present invention furthermore includes a method in which a consistency check of the respiratory rates fᵢ(n) (i=1, 2, . . . ) is carried out. Defective signals can thus be identified and suppressed in the determination of the respiratory rate f. In the method described above, this is advantageously done before the weighted averaging of the respiratory rates fᵢ(n) (i=1, 2, . . . ). It is, however, obvious to the person of average skill in the art that such a consistency check is also of great advantage independently of the specific averaging.

The consistency check advantageously takes place by a comparison of the respiratory rates fᵢ(n) (i=1, 2, . . . ) among one another. This allows a check of the consistency of the individual respiratory rates fᵢ(n) (i=1, 2, . . . ) in a simple manner such that inconsistent values can be sorted out and the quality of the signal can be determined from these differences. The more agreements that are found between the different respiratory rates fᵢ(n) (i=1, 2, . . . ) in the consistency check, the higher the signal quality is assessed.

Advantageously, the difference between the respective respiratory rates fᵢ(n) (i=1, 2, . . . ) is still compared with a permitted tolerance Δ. Minor differences in the consistency check are thus ignored, while large differences indicate an inconsistency between the individual values of the respiratory rates fᵢ(n) (i=1, 2, . . . ).

Advantageously, only those respiratory rates which pass the consistency test are used for the weighted averaging of the respiratory rates fᵢ(n) (i=1, 2, . . . ). Errors can thus be suppressed right from the start and no longer influence the final result. The signal quality can moreover be assessed from the number of respiratory rates passing the consistency check.

Further advantageously, the present invention comprises a method in which the signal quality is in particular determined via a consistency check as described above and is optionally displayed. It is obvious to the skilled person in this connection that such a determination of the signal quality delivers important information for the evaluation of the measured results and is also of great advantage independently of the features of the method described above.

The present invention furthermore includes a method comprising the following steps: the generation of at least two frequency signals FTᵢ(f) (i=1, 2, . . . ) by transformation of at least two time dependent respiratory signals sᵢ(t) (i=1, 2, . . . ) into the frequency domain as well the determination of a frequency signal FT(f) by a combination of the frequency signals FTᵢ(f) (i=1, 2, . . . ), wherein a respiratory rate f is determined on the basis of the frequency signal FT(f). The transformation of the time dependent respiratory signals sᵢ(t) (i=1, 2, . . . ) into the frequency domain can take place by a Fourier transform and advantageously by a fast Fourier transform (FFT). The frequency spectra of the different respiratory signals thus result which can then be used for determining the frequency signal FT(f). This also makes possible a simple and reliable suppression of interference signals and errors in the final result. It is obvious to the skilled person in this connection that this method is a method which is independent of the averaging in time and space described above and which can, however, advantageously be combined with this e.g. for the initializing of the weighted averaging or for the bridging of strong interference.

In this combination, the frequency signal FT(f) is advantageously determined in the combination of the frequency signals by an averaging of the frequency signals FTᵢ(f) (i=1, 2, . . . ). The geometric average is advantageously calculated in this process.

The respiratory rate f is now advantageously determined by peak detection of the frequency signal FT(f) so that the average respiratory rate f can be derived directly from the frequency signal.

Alternatively, the respiratory rate f can, however, also be determined by back transformation of the frequency signal FT(f) and an evaluation of the resulting signal s(t). This evaluation can then take place, as already described above, by a determination of the maxima of the signal s(t).

Two simple methods are thus available to determine the respiratory rate f from the frequency signal FT(f).

Further advantageously, in the method in accordance with the invention, the at least two time dependent respiratory signals sᵢ(t) (i=1, 2, . . . ) are acquired from a PPG signal and an ECG signal. These two signals contain a plurality of information on the respiratory rate and thus form a reliable basis for determining the at least two time dependent respiratory signals sᵢ(t) (i=1, 2, . . . ) by different methods.

Advantageously, the at least two time dependent respiratory signals sᵢ(t) (i=1, 2, . . . ) of the method in accordance with the invention form a selection from the following signals:
a respiratory signal S_{HR}(t) determined from the heart rate,
a respiratory signal S_{PPG}(t) determined from the PPG signal,
a respiratory signal s_{PTT}(t) determined from the PTT signal,
a respiratory signal Sₖᵤᵣₜ(t) determined from the kurtosis of the ECG signal.

All these respiratory signals can then be evaluated and utilized for determining the respiratory rate f of the patient.

In this connection, all four respiratory signals are advantageously used in the method in accordance with the invention to achieve a reliability and precision of the result which is as high as possible. A high number of respiratory signals is in particular of advantage when using the consistency check and the determination of the signal quality.

The present invention furthermore includes an apparatus for determining the respiratory rate of a patient by means of one of the methods described above. The same advantages hereby obviously result as have already been presented with respect to the method. Such an apparatus in particular includes sensors for measuring physiological signals from which the at least two time dependent respiratory signals can be determined as well as a means for data processing which are designed out such that they perform the method in accordance with the invention.

Further advantageously, the present invention includes an apparatus for determining the respiratory rate of a patient, in particular for the carrying out of the method in accordance with the invention, comprising a sensor unit for the measurement of the physiological signals from which the at least two time dependent respiratory signals can be determined and a processing unit for the evaluation of the data transmitted by the sensor unit. Since at least a large part of the method for determining the respiratory rate of a patient is not carried out in the sensor unit, but in the processing unit, the processing power of the sensor unit required for the carrying out of the method steps performed in the sensor unit does not have to be dimensioned all that large, which permits a cost-effective and space-saving design. A particularly simple operation of the apparatus in accordance with the invention is possible by the separate sensor unit, with particular advantages in particular resulting when using the method in accordance with the invention. It is, however, anyway obvious to the skilled person that advantages likewise result on using a method in accordance with the prior art.

Further advantageously, the data generated by the sensor unit are transmitted to the processing unit in a wireless manner. No complicated wiring is hereby required, which in turn increases the user friendliness and the operating security of the apparatus in accordance with the invention.

Further advantageously, the sensor unit is fastened to the wrist of the patient. Such a sensor unit formed e.g. as a wrist device permits a particularly simple operation which is also less of a strain for the patient. Any known type of wireless transmission can be used for the data transmission, with a radio transmission of the data in particular being of advantage. In this connection, the data are transmitted in a wireless manner from the sensor unit to the processing unit which is e.g. arranged in a device for the treatment or for the monitoring of the patient.

Parts of the method for determining the respiratory rate can already be carried out in the sensor unit so that further processed data are transmitted to the processing unit. A certain processing power must thus admittedly be made available in the sensor unit, but the data amounts to be transmitted from the sensor unit to the processing unit are accordingly smaller so that the data transmission means from the sensor unit to the processing unit can be dimensioned in a less costly and/or complex manner. This in particular has substantial advantages on the use of wireless transmission.

The at least two time dependent respiratory signals are advantageously determined from the physiological signals in the sensor unit and are thereupon transmitted to the processing unit. The evaluation by means of band pass and the subsequent steps of the method in accordance with the invention then take place by the electronic system of the processing unit.

It is naturally also possible to carry out further steps of the method in accordance with the invention in the sensor unit, with it having to be noted here, however, that a specific processing power (processor power) is required for the further evaluation so that expensive and/or complex hardware is preferably not arranged in the sensor unit, but rather in the processing unit. The interface can, however, generally be selected as desired.

Further advantageously, the apparatus in accordance with the invention comprises sensors for the measurement of the ECG signal and of the PPG signal. The at least two time dependent respiratory signals of the method in accordance with the invention can be determined from these two physiological signals, with any errors in the individual signals being able to be eliminated by the averaging in accordance with the invention. Further advantageously, the heart rate, the pulse amplitude and the pulse wave transit time are determined from the ECG signal and the PPG signal. Three different time dependent respiratory signals are hereby available by whose averaging in accordance with the invention systematic errors in the output signals can also be eliminated.

The processing unit is advantageously part of a medical device, in particular of a medical device for the extracorporeal treatment of blood such as a dialysis machine, a hemofiltration machine or a hemodiafiltration machine. The data transmission and a further evaluation of the data in accordance with the invention can, however, naturally also take place in connection with any other desired medical device.

Alternatively, the processing unit of the apparatus in accordance with the invention can also be part of a computer network, e.g. of a hospital or of a dialysis clinic. This has the advantage that the expensive and/or complex hardware for the evaluation of the data transmitted by the sensor unit can be accommodated in the computer network of the hospital or dialysis clinic.

The present invention will now be described in more detail with reference to drawings.

There are shown:

FIG. 1: four extracted respiratory signals and a respiratory signal measured with a thermistor;

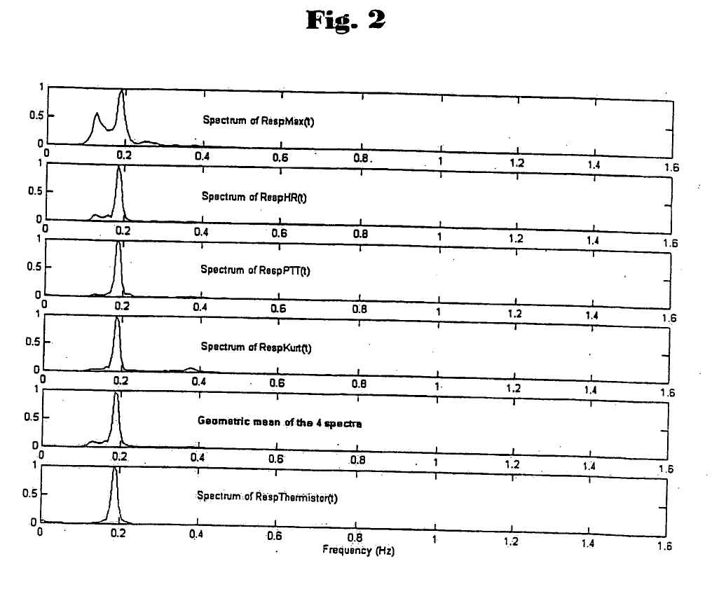

FIG. 2: frequency spectra of the four extracted respiratory signals, the geometric average of the four frequency spectra and the frequency spectrum of the thermistor signal;

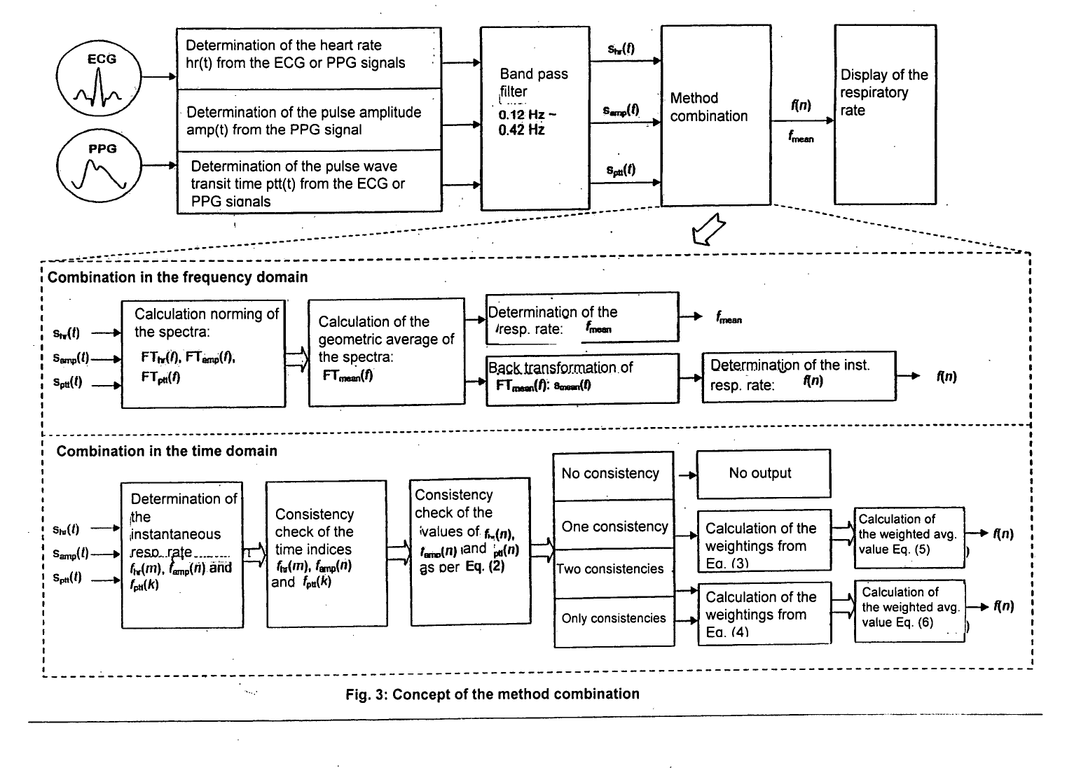

FIG. 3: the structure of an embodiment of the method combination in accordance with the invention;

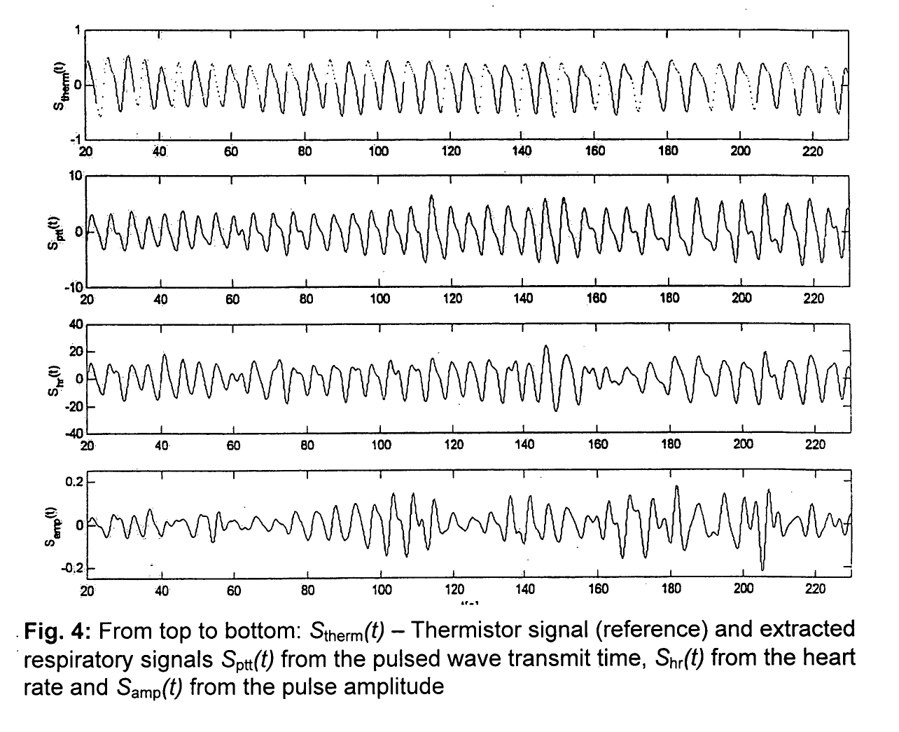

FIG. 4: a respiratory signal measured with the thermistor as a reference and three extracted respiratory signals; and

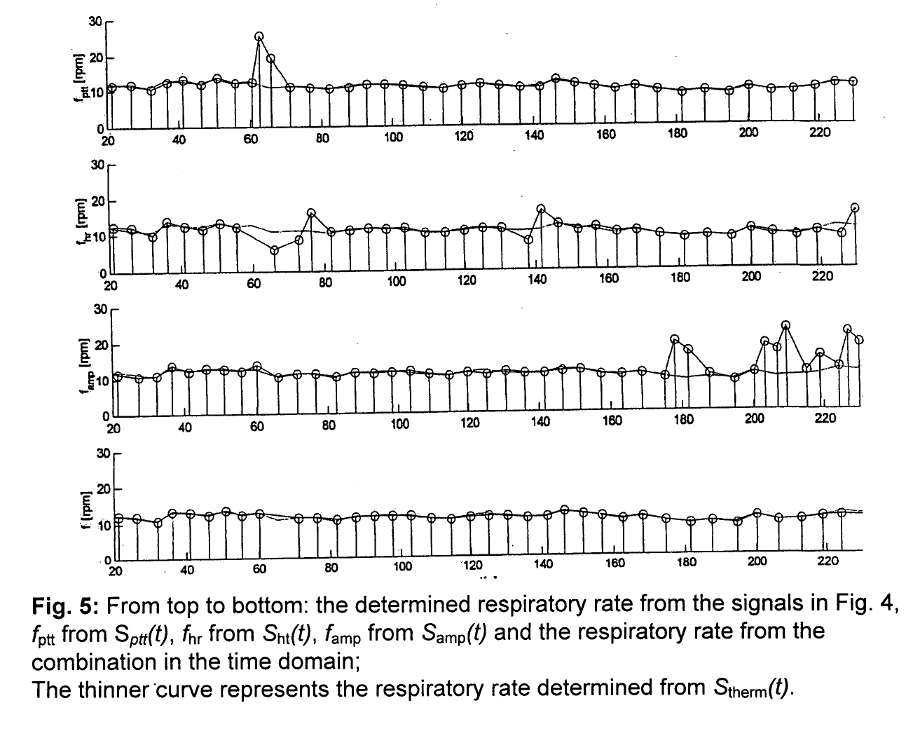

FIG. 5: the respiratory rates determined from individual channels as well as the respiratory rate determined in accordance with the invention from the combination in comparison with the respiratory rate from the thermistor signal.

The following methods for indirect respiratory monitoring are known from the prior art in addition to the direct respiratory monitoring via a thermistor which is, however, felt to be very irritating by the patients:
Respiratory Monitoring via the Bioimpedance Measurement [1]
   The thorax expands and the impedance increases on inhaling. On exhaling, the thorax contracts and the impedance falls. If a constant alternating current is conducted through the thorax, a respiratory dependent voltage can be measured via two ECG electrodes.
Respiratory activity from the heart rate variability signal [2]
Respiratory activity from the photoplethysmographic signal (PPG signal) [3]
Respiratory activity from the source statistics of the ECG [4]
Respiratory activity from the pulse wave transit time [5]

In the embodiment of the present invention, an improvement of the reliability of the respiratory information extracted from the ECG signal and the PPG signal is achieved by the combination of the known methods in either the time domain or the frequency domain.

### 2. Physiological Principles

It will be explained in the following why, from a physiological aspect, the ECG signal and the PPG signal contain information on respiration.

### 2.1 Respiratory Sinus Arrhythmia (RSA)

The dependence of the heart rate on the respiration is known as respiratory sinus arrhythmia (RSA).
   An increase in the heart rate during inspiration
      A fall in the heart rate during expiration
The RSA is above all communicated by the changing activity of the vagus nerve. Respiratory sinus arrhythmia can thus be interrupted by dispensing atropine or vatogomy.
Influences on the respiration-dependent heart rate variability: e.g. pulmonary, vascular and cardiac stretch receptors and respiratory centers in the brainstem, different baroreflex sensitivity in the respective phases of the respiratory cycle.
Due to an inspiratory vagal inhibition, fluctuations in the heart rate result at the same frequency as respiration.
The inspiratory inhibition is primarily caused by the influence of the medullar respiratory center on the medullar cardiovascular center.
In addition, peripheral reflexes are responsible due to hemodynamic changes and thoracic stretch receptors.
Fluctuations of the blood pressure (Traube-Hering waves) are also accordingly known of the same frequency.

Other periodic fluctuations of the heart rate, in addition to respiratory sinus arrhythmia, are the baroreceptor reflex heart rate changes and the thermoregulatory induced heart rate changes:
The so-called 10-second rhythm of the heart rate is caused by self-oscillations of the vasomotoric part of the baroreflex loop.
These intrinsic oscillations result from the negative baroreflex feedback system and are accompanied by synchronous fluctuations of the blood pressure (Mayer waves).
The frequency of these fluctuations is determined by the time delay of the system which increases with an increased sympathetic tone and decreases with sympathetic or parasympathetic blockades.

The peripheral resistance shows intrinsic oscillations at a low frequency.
These fluctuations can be caused by a thermal stimulation of skin and are thus considered a reaction to thermoregulatorily necessary changes in the dermal blood flow.
These periodic changes in the peripheral resistance are accompanied by oscillations of the blood pressure and of the heart rate.

### 2.2 Respiratory Induced Fluctuations in the Blood Pressure

The blood pressure fluctuates by an average value in dependence on respiration. Mechanical effects of the respiration on the blood pressure are presumed to be the cause. Mayer found further blood pressure oscillations whose frequencies were lower than those of the respiration. They arise due to changes in the peripheral vascular tone with a periodicity of approx. 10-20 sec. (0.1 Hz) and are called “Mayer waves”. The physiological blood pressure changes are divided into fluctuations of I, II and III order:

I order: Change by systole and diastole;

II order: Changes in dependence on the respiration; and

III order: Mayer waves (0.1 Hz).

In addition, blood pressure fluctuations of a lower frequency (<0.04 Hz) are known.

In the following table, the fluctuations in the blood pressure are summarized with the corresponding causes:

TABLE 1
Rhythm in the blood pressure and possible cause

| Blood | | |
|---|---|---|
| pressure | Frequency | |
| fluctuation | domain (Hz) | Possible causes |
| I order | 0.5~2.0 | Cardiac contraction |
| II order | 0.15~0.40 | Respiration - mechanical effects of the |
| | | respiration on the blood pressure |
| III order | 0.04~0.15 | Mayer waves - The sympathetic nervous |
| | | system communicates part of these |
| | | fluctuations. The LF power is subject to |
| | | regulation by baroreflex and homoral |
| | | influences. |
| | <0.04 | The oscillations reflect the interaction |
| | | between different control mechanisms, e.g. |
| | | of thermoregulation and of the renin- |
| | | angiotensin system, of the endothelial |
| | | function. |

### 3. Extraction of the Respiratory Activity from the PPG and ECG

### 3.1 Respiratory Activity from the Heart Rate Signal

The ECG signal and PPG signal are frequency modulated by the respiration due to the respiratory sinus arrhythmia. In this respect, the PPG signal is given by

PPG(t)=PPG(ω_{Herz}s(ω_{Resp}·t)·t),

where

ω_{Herz} is the heart rate and

s(ω_{Resp}·t) is the respiratory signal with the respiratory rate ω_{Resp}.

The frequency modulation can be demodulated by the respiration in that, first, the instantaneous heart rate is determined from the ECG signal or from the PPG signal on a “beat-to-beat” basis. Then the heart rate variability signal and thus the temporal respiratory signal s_{HR}(t) is extracted with the help of a band pass filter of 0.12 Hz-0.42 Hz.

### 3.2 Respiratory Activity from the PPG Signal

The respiratory activity is taken into the PPG signal in the form of an additive signal portion as a consequence of respiratory induced fluctuations in the blood pressure. The respiratory rhythm is reflected in the PPG signal and is represented by

PPG(t)=PPG(ω_{Herz}·s(ω_{Resp}·t)·t)+k_{ppg}·s(ω_{Resp}·t),

where k_{ppg} is the strength of the additive characteristic of s(ω_{Resp}·t) in the PPG signal.

To acquire the additive respiratory signals, the envelope of the PPG signal can first be formed by the “beat-to-beat” determination of the local maxima or minima in the PPG signal and then the temporal respiratory signal s_{PPG}(t) can be extracted using the band pass filter.

### 3.3 Respiratory Activity from the PTT Signal

Since the fluctuation in the blood pressure has a respiratory induced portion, on the one hand, and the systolic blood pressure correlates in an almost linear fashion with the PTT, respiratory information is also contained in the PTT. This has the effect that the PTT has an additive respiratory portion. The PTT signal can therefore be given by

PTT(t)=PTT_{sBP}(t)+kₚₜₜ·s(ω_{Resp}·t),

where

PTT_{sBP}(t) is the systolic blood pressure induced portion in the PTT and

kₚₜₜ is the strength of the additive characteristic of s(ω_{Resp}·t) in the PTT signal.

Respiratory activity can be extracted from the PTT signal with the help of the band pass filter.

### 3.4 Respiratory Activity from the Kurtosis of the ECG

The basis of this method is formed by the assumption that the transmission path of the electrical signals from the heart via the thorax up to the surface of the skin can be considered as a linear, time-variant system whose properties are predetermined by the state of the body. One property of the system in this connection is the impedance of the thorax which is changed by the respiration. These time variations of the system should be made visible by the kurtosis. The kurtosis value is calculated using the following formula:

$$Kurtosis = \frac{1}{T} {\sum }_{t = 1}^{T} {[ \frac{{x}_{1} - \overline{x}}{\sqrt{\frac{1}{T}} {\sum }_{t = 1}^{T} {( {x}_{1} - \overline{x} )}^{2}} ]}^{4}$$

The procedure for the extraction of the respiratory rhythm from the ECG using the kurtosis method can be divided into the following steps:
1. Elimination of the baseline drift in the ECG signal;
2. Location of the R-spikes: The ECG signal development between two successive R-spikes forms an interval;
3. Kurtosis calculation: The kurtosis is calculated for each defined interval using the formula given above and is stored with the associated point in time;
4. Formation of an envelope via the calculated kurtosis values;
5. The temporal respiratory signal sₖᵤᵣₜ(t) arises by filtering the envelope using the band pass filter.

### 4. Method Combination

As already initially mentioned, not only the respiratory rhythm is characterized in the blood pressure and in the heart rate, but also other interference rhythms such as Mayer waves and fluctuations by the vascular tone and the thermoregulation which are in the frequency domain from 0.0 Hz˜0.15 Hz. Since such interference rhythms are partly superimposed on the respiratory rhythm in the frequency domain, they can also be present in the respiratory signals extracted from the PPG and the ECG. The respiratory measurement can thereby be falsified.

Due to the complexity and difference of the transmission paths, the interference rhythms can be characterized differently in the extracted respiratory signals sₕᵣ(t), sₘₐₓ(t), sₚₜₜ(t), sₖᵤᵣₜ(t). FIG. 1 and FIG. 2 show four such respiratory signals in the time and frequency domains.

The signal evaluation furthermore shows that the characterizations of the interference rhythms in the four respiratory signals are person-dependent and vary in time. For this reason, it is usually difficult to judge the quality of the extracted respiratory signals. For example, it is not possible to simply state that sₕᵣ(t) is definitively better or worse than sₚₜₜ(t).

The basic idea of the method combination in the time or frequency domains is based on the aforesaid observation. It serves the increase in reliability of the respiratory information extracted from the ECG and the PPG.

To be able to combine e.g. four different methods, the 2 following steps must first be taken:
detection of the ECG and PPG signals for a predetermined time duration T and determination of the heart rate hr(t), of the PPG maximum max(t), of the pulse wave transit time ptt(t) and of the kurtosis value kurt(t) on a “beat-to-beat” basis.
Filtering of the four signals using the bandpass 0.12 Hz˜0.42 Hz. The four corresponding respiratory signals sₕᵣ(t), sₘₐₓ(t), sₚₜₜ(t) and sₖᵤᵣₜ(t) result from this.

### 4.1 Combination in the Time Domain

### 4.1.1 Determining the Instantaneous Respiratory Rate

locate local maxima and store their time index tₘₐₓ(n) in seconds
calculating the respiratory rate using:

$$f ( n ) = \frac{60 sec}{{t}_{ma x} ( n ) - {t}_{ma x} ( n - 1 )}$$

in breaths/min
determining the instantaneous respiratory rate from the four respiratory signals
   fₕᵣ(n) from sₕᵣ(t)
   fₘₐₓ(n) from sₘₐₓ(t)
   fₚₜₜ(n) from sₚₜₜ(t)
   fₖᵤᵣₜ(n) from sₖᵤᵣₜ(t)

### 4.1.2 Combination by Weighted Averaging

The 4 measured respiratory rates are first compared with an estimate of the current respiratory rate and their differences from the estimate are calculated for a weighted averaging. The calculation of the weight factors in dependence on the differences then takes place. The larger the difference, the smaller the weight factor. Last, a final respiratory rate is fixed by the weighted averaging.

The weighted averaging will be described in more detail in the following, with the last respiratory rate being considered as the estimate of the current respiratory rate.
1. Calculation of the difference of the instantaneous respiratory rate from the last respiratory rate f(n−1):

σₕᵣ²=[fₕᵣ(n)−f(n−1)]²

σₘₐₓ²=[fₘₐₓ(n)−f(n−1)]²

σₚₜₜ²=[fₚₜₜ(n)−f(n−1)]²

σₖᵤᵣₜ²=[fₖᵤᵣₜ(n)−f(n−1)]²
2. Calculation of the weight factor:

$${k}_{hr} = \frac{Σ - {σ}_{hr}^{2}}{3 \cdot Σ}$$

$${k}_{ma x} = \frac{Σ - {σ}_{ma x}^{2}}{3 \cdot Σ}$$

$${k}_{ptt } = \frac{Σ - {σ}_{ptt}^{2}}{3 \cdot Σ}$$

$${k}_{kurt} = \frac{Σ - {σ}_{kurt }^{2}}{3 \cdot Σ}$$

where Σ=σₕᵣ²+σₘₐₓ²+σₚₜₜ²+σₖᵤᵣₜ²
3. Calculation of the current respiratory rate f(n) by weighted averaging according to:

f(n)=fₕᵣ(n)·kₕᵣ+fₘₐₓ(n)·kₘₐₓ+fₚₜₜ(n)·kₚₜₜ+fₖᵤᵣₜ(n)·kₖᵤᵣₜ
4. Initialization f(0)
initializing using a fixed value, e.g. 12 breaths/min—normal respiratory rate for adults:

f(0)=12 breaths/min
initializing using the arithmetic mean of the instantaneous respiratory rates:

$$f ( 0 ) = \frac{1}{4} \cdot [ {f}_{hr} ( 0 ) + {f}_{ma x} ( 0 ) + {f}_{ptt} ( 0 ) + {f}_{kurt} ( 0 ) ]$$

f(0) results from a respiratory rate determined with the help of the combination in the frequency domain.
5. Table 2 shows some examples of the weighted averaging

TABLE 2
Examples for the weighted averaging

| Last value | | | | | Weighted | Arithmetic |
|---|---|---|---|---|---|---|
| f(n − 1) | fₕᵣ(n) | fₘₐₓ(n) | fₚₜₜ(n) | fₖᵤᵣₜ(n) | average f(n) | mean |
| 12 | 15 | 15 | 15 | 15 | 15.0 | 15.0 |
| 12 | 12 | 13 | 11 | 8 | 11.9 | 11.0 |
| 12 | 13 | 11 | 6 | 8 | 10.6 | 9.5 |
| 12 | 11 | 8 | 7 | 6 | 8.4 | 8.0 |
| 12 | 9 | 8 | 7 | 4 | 7.5 | 7.0 |

### 4.1.3 Combination by Consistency Check—“Consensus Method”

The four respiratory rates of fₕᵣ(n), fₘₐₓ(n), fₚₜₜ(n) and fₖᵤᵣₜ(n) are checked among one another for consensus while taking account of a predetermined tolerance. Then, in dependence on the number of consensus points, a final respiratory rate is calculated via arithmetic or weighted averaging from the respiratory rates with consensus. The more consensus points there are, the more reliable the final respiratory rate.

The consensus check is described in more detail as follows.
1. A tolerance Δ is defined as a permitted deviation for the check of consensus of the respiratory rates. fₕᵣ(n), fₘₐₓ(n), fₚₜₜ(n) and fₖᵤᵣₜ(n), e. g. Δ=2 breaths/min.
   The tolerance Δ can be dependent on the past measured data. For example, it can be dependent on the last instantaneous respiratory rate and/or on an average respiratory rate.
2. Calculation of the difference of two respiratory rates according to

Δₖ₋₁=|fₖ(n)−f₁(n))|

Calculation of a consistency factor according to
consistent: αₖ₋₁=1, when Δₖₗ≦Δ
non-consistent: αₖ₋₁=0, when Δₖₗ>Δ

A total of 6 consistency factors thus result which are summarized in Table 3:

TABLE 3
Consistency factors

| | fₕᵣ(n) | fₘₐₓ(n) | fₚₜₜ(n) | fₖᵤᵣₜ(n) |
|---|---|---|---|---|
| fₕᵣ(n) | 1 | αₕᵣ₋ₘₐₓ | αₕᵣ₋ₚₜₜ | αₕᵣ₋ₖᵤᵣₜ |
| fₘₐₓ(n) | | 1 | α_{ppg-ppt} | α_{ppg-kurt} |
| fₚₜₜ(n) | | | 1 | αₚₜₜ₋ₖᵤᵣₜ |
| fₖᵤᵣₜ(n) | | | | 1 |

3. At least two of four respiratory rates must be consistent to be able to determine a respiratory rate. The determination of the final respiratory rate takes place via a weighed averaging.

### 4.2 Combination in the Frequency Domain

The formation of a geometrically averaged spectrum is the central point of the combination in the frequency domain. The interference rhythms in the signals should thereby be fully or partly eliminated. This method is based on the observation that, on the one hand, the interference rhythms have very different characteristics and, on the other hand, the respiratory rhythm are reflected relatively consistently in the extracted respiratory signals of sₕᵣ(t), sₘₐₓ(t), sₚₜₜ(t) and sₖᵤᵣₜ(t).

The method combination in the frequency domain takes place via:
1. The signals of sₕᵣ(t), sₘₐₓ(t) sₚₜₜ(t) and sₖᵤᵣₜ(t) for a given time interval are transformed by e.g. FFT (“fast Fourier transformation”) into the frequency domain and subsequently formed. The corresponding spectra of FTₕᵣ(f), FTₘₐₓ(f), FTₚₜₜ(f) and FTₖᵤᵣₜ(f) result from this.
2. The geometric average of the spectra is calculated by:

FTₘₑₐₙ(f)=[FTₕᵣ(f)·FTₘₐₓ(f)·FTₚₜₜ(f)·FTₖᵤᵣₜ(f)]^{1/4}
3. Determination of an average respiratory rate from FTₘₑₐₙ(f) by
   a) e.g. peak detection or
   b) the averaged spectrum FTₘₑₐₙ(f) is transformed back into the time domain A temporal respiratory signal sₘₑₐₙ(t) results from this which is partly or fully free of interference rhythms. The instantaneous respiratory rate can be determined from sₘₑₐₙ(t) in accordance with the method described in section 4.1.1.

In comparison with the combination in the time domain, the combination in the frequency domain has the disadvantage that more calculation and time effort has to be taken up.

### 4.3. A Specific Embodiment

In the specific embodiment of the method combination in accordance with the invention, signals from three different channels are combined, with all three combination methods described above, i.e. the combination by weighted averaging, by a consistency check and by an averaging in the frequency domain, being used. A diagram of this embodiment can be seen in FIG. 3.

Extraction of the Respiratory Information from the ECG and PPG
1. Detection of the ECG signal and the PPG signal for a predetermined time period T and determination of the following three respiratory signals:
   rr(t) or pp(t)—RR distance from the ECG or “peak-to-peak” distance from the PPG
   amp(t)—pulse amplitude from the PPF signal
   ptt(t)—pulse wave transit time from the PPG signal and the ECG signal
2. Filtering of the three signals using the band pass filter from 0.12 Hz˜0.42 Hz. There result from this
sₕᵣ(t)—respiratory signal from the variation of the heart rate rr(t) or pp(t)
sₐₘₚ(t)—respiratory signal from the variation of the pulse amplitude amp(t)
sₚₜₜ(t)—respiratory signal from the variation of the pulse transit time ptt(t)

Combination in the Frequency Domain

The formation of the geometrically averaged spectrum is the central point in the combination in the frequency domain. The interference rhythms which are within the frequency domain (0.12 Hz-0.42 Hz) of the band pass filter and thus cannot be eliminated by the filter should thereby be fully or partly eliminated in the extracted respiratory signals. This method is based on the observation that, on the one hand, the interference rhythms have very different characteristics and, on the other hand the respiratory rhythm is characterized relatively consistently in the extracted respiratory signals of sₕᵣ(t), sₐₘₚ(t) and sₚₜₜ(t).

The method combination in the frequency domain will be explained with reference to FIG. 3, for example for sₕᵣ(t), sₐₘₚ(t) and sₚₜₜ(t). It is done via:
1. The signals of sₕᵣ(t), sₐₘₚ(t) and sₚₜₜ(t) for a given time interval are transformed by e.g. FFT (“fast Fourier transformation”) into the frequency domain and subsequently normed. The corresponding spectra of FTₕᵣ(f), FTₐₘₚ(f) and FTₚₜₜ(f) result from this.
2. The geometric average of the spectra is calculated according to:

FTₘₑₐₙ(f)=[FTₕᵣ(f)·FTₐₘₚ(f)·FTₚₜₜ(f)]^{1/3} (1)
3. Determination of an average respiratory rate from FTₘₑₐₙ(f) by e.g. peak detection or
4. The averaged spectrum FTₘₑₐₙ(f) is transformed back into the time domain.

A temporal respiratory signal sₘₑₐₙ(t), results from this which is partly or fully free of interference rhythms.
5. The instantaneous respiratory rate can be determined from sₘₑₐₙ(t) using the method described in section 4.1.1.

Combination in the Time Domain
1. Determining the instantaneous respiratory rate in breaths/min:
   fₕᵣ(m) from sₕᵣ(t)
   fₐₘₚ(n) from sₐₘₚ(t)
   fₚₜₜ(k) from sₚₜₜ(t)
2. Consistency check for the time indices of m, n and k:
They must be within a predetermined time window provided they belong to a respiratory activity or a breath. 50% of the current respiratory period can, for example, be used for the time window. If the test is passed, the respiratory rates are again termed fₕᵣ(n), fₐₘₚ(n) and fₚₜₜ(n).
3. Consistency check for the values of the respiratory rates of
   fₕᵣ(n), fₐₘₚ(n) and fₚₜₜ(n) according to:

|f_{A}(m)−f_{B}(m)|≦th (2)
where A,B=hr, amp, ptt
For example th=2.5 breaths/min or th=15% of the last respiratory rate
Continuation after the test result:
a) No consistency CP=0
b) One consistency CP=1, e.g. only for fₐₘₚ(n) und fₚₜₜ(n)
c) Two consistencies: CP=2, e.g. for both fₐₘₚ(n) und fₚₜₜ(n) and fₐₘₚ(n) und sₚₜₜ(n)
4. Calculation of the weight factors based on the last respiratory rate from the combination.
   a) Case 1: CP=0
      No weight factor is calculated.
   b) Case 2: CP=1

$${k}_{amp} = \frac{Σ - {e}_{amp }^{2}}{Σ} \\ {k}_{ptt} = \frac{Σ - {e}_{ptt}^{2}}{Σ} \\ {e}_{amp} = {f}_{amp} ( n ) - f ( n - 1 ) \\ {e}_{ptt} = {f}_{ptt} ( n ) - f ( n - 1 ) \\ Σ = {e}_{amp}^{2} + {e}_{ptt}^{2} \qquad ( 3 )$$

where f(n−1)—last valid respiratory rate from the combination
c) Case 3: CP=2

$${k}_{hr} = \frac{Σ - {e}_{hr}^{2}}{2 \cdot Σ} \\ {k}_{amp} = \frac{Σ - {e}_{amp}^{2}}{2 \cdot Σ} \\ {k}_{ptt} = \frac{Σ - {e}_{ptt}^{2}}{2 \cdot Σ} \\ {e}_{hr} = {f}_{hr} ( n ) - f ( n - 1 ) \\ {e}_{amp} = {f}_{amp} ( n ) - f ( n - 1 ) \\ {e}_{ptt} = {f}_{ptt} ( n ) - f ( n - 1 ) \\ Σ = {e}_{ht}^{2} + {e}_{amp}^{2} + {e}_{ptt}^{2} \qquad ( 4 )$$

5. Weighted averaging
   a) Case 1: CP=0
      No averaging possible=>No output of the respiratory rate
   b) Case 2: CP=1

f(n)=kₐₘₚ·fₐₘₚ(n)+kₚₜₜ·fₚₜₜ(n) (5)
c) Case 3: CP=2

f(n)=kₕᵣ·fₕᵣ(n)+kₐₘₚ·fₐₘₚ(n)+kₚₜₜ·fₚₜₜ(n) (6)
6. Initialization—determination of the first value of the respiratory rate f(0)
   Possibility 1
      With a passed consistency check (CP≧1), f(0) is calculated as the arithmetic mean of the consistent respiratory rates
   Possibility 2
      The method combination is carried out in the frequency domain and takes the average respiratory rate determined therefrom as f(0).

Result

FIG. 4 shows, from top to bottom, the thermistor signal sₜₕₑᵣₘ(t) (reference), the extracted respiratory signals of sₚₜₜ(t) from the pulse wave transit time, sₕᵣ(t) from the heart rate and sₐₘₚ(t) from the pulse amplitude. FIG. 5 shows the respiratory rates determined from the signals shown in FIG. 4 and the respiratory rate from the combination in the time domain. The thin curves in FIG. 5 show the respiratory rate from the thermistor signal.

It can clearly be recognized from FIG. 5 that the individual respiratory rates from the respective extracted respiratory signals differ at some points from the respiratory rates from the thermistor signal, e.g. fₚₜₜ between 60 s and 70 s; fₕᵣ between 60 s and 80 s, by 140 s, after 220 s; fₐₘₚ by 180 s, after 200 s. Fully in contrast, the respiratory rate from the combination has a very good consensus with the respiratory rate from the thermistor signal. It can also be recognized that the interference of the respiratory rate between 60 s and 70s is eliminated by the combination. The reason for this is the consistency check which the disrupted signals have not passed.

### 4.4 Generality of the Method Combination

The aforesaid method combination is not restricted to signals of sₕᵣ(t), sₘₐₓ(t), sₚₜₜ(t) and sₖᵤᵣₜ(t). It can be used both for respiratory signals extracted from the ECG signal and/or the PPG signal and for respiratory signals detected with other sensors/methods (e.g. thermistor, impedance pneumography, induction plethysmography).

The different alternatives of the method combination such as the weighted averaging, the consistency check and the combination in the frequency domain can in turn also be combined with one another.

The initially mentioned different methods for the determination of the respiratory rate are shown in the following publications whose content is included in the present application by reference:
[1] Association of the Advancement of Medical Instrumentation (AAMI): Apnea Monitoring by Means of Thoracic Impedance Pneumography, AAMI, Arlington, Va., 1989,
[2] Hirsch J A, Bishop B.: Respiratory Sinus Arrhythmia in Humans: How breathing pattern modulates Heart rate, Am J Physiol. October 1981; 241 (4): H620-9
[3] Anders Johansson, Per Ake Öberg and Gunnar Sedin: Monitoring of Heart and Respiratory Rates in Newborn Infants using a new Photoplethysmographic Technique, Journal of Clinical Monitoring and Computing, 15: 461-467, 199
[4] Shuxue Ding, Xin Zhu, Wenxi Chen und Darning Wei: Derivation of Respiratory Signal from Single-Channel ECG Based on Source Statistics, International Journal of Bioelectromagnetism, Vol. 6, No. 1, 2004
[5] Wei Zhang, D E 10014077A1 “A Method and an Apparatus for Determining Breathing Activity for a Human or Other Organism”, date of application March 2, 2000

Abbreviations:
ECG: electrocardiogram—recording of cardiac activity by the detection of the potential differences at the surface of the body dependent on cardiac excitation.
PPG: photoplethysmogram—record of the blood volume using an optoelectronic measuring method
PTT: pulse wave transit time—the time a pulse wave takes to move along an artery from a position A (close to the heart) to a (peripheral) position B.
RSA: respiratory sinus arrhythmia—respiration induced change in the heart rate
Resp: Respiration
sBP: systolic blood pressure
HR: heart rate
FFT: “fast Fourier transformation”

1. A method of determining the respiratory rate of a patient comprising the steps:
determining at least two time dependent respiratory signals sᵢ(t) (i=1,2, . . . ) by at least two different methods;
determining the resulting respective instantaneous respiratory rates fᵢ(n) (i=1,2, . . . ) from the at least two time dependent respiratory signals sᵢ(t) (i=1, 2, . . . )
determining an average respiratory rate f(n) by a weighted averaging of the respiratory rates fᵢ(n) (i=1,2, . . . ), characterized in that
the weightings kᵢ(n) (i=1,2, . . . ) of the individual respiratory rates fᵢ(n) (i=1,2, . . . ) depend on a difference between the respective respiratory rates fᵢ(n) (i=1,2, . . . ) and an estimate fₛ(n) determined on the basis of at least two respiratory signals sᵢ(t) (i=1,2, . . . ).

2. A method in accordance with claim 1, wherein the estimate fₛ(n) is determined on the basis of a preceding, already determined average respiratory rate f(n−1).

3. A method in accordance with claim 1, wherein the estimate fₛ(n) is determined, in particular for initialization by a combination of rate information from at least two time dependent respiratory signals sᵢ(t) (i=1,2, . . . ) or by forming an average of the current respiratory rates fᵢ(n) (i=1, 2, . . . ).

4. A method in accordance with claim 1, wherein the at least two time dependent respiratory signals sᵢ(t) (i=1,2, . . . ) are determined from measured physiological signals.

5. A method in accordance with claim 4, wherein the measured physiological signals form a selection from the following signals:
bioimpedance signal;
heart rate variability signal;
photoplethysmographic signal (PPG signal);
statistical source signal of the ECG;
pulse wave transit time signal (PTT signal).

6. A method in accordance with claim 4, wherein the determination of the at least two time dependent respiratory signals sᵢ(t) (i=1,2, . . . ) takes place from the measured physiological signals by a band pass filter.

7. A method in accordance with claim 6, wherein the band pass filter allows frequencies in a range from approx. 0.12 Hz to 0.42 Hz to pass.

8. A method in accordance with claim 1, wherein the determination of the respective instantaneous respiratory rates fᵢ(kᵢ), in particular of fₕᵣ(m), fₐₘₚ(n) and fₚₜ(k), from the time dependent respiratory signals sᵢ(t) (i=1,2, . . . ) takes place by determining the time indices tₘₐₓ(kᵢ), in particular tₘₐₓ(m), tₘₐₓ(n) and tₘₐₓ(k), of the maxima of the time dependent respiratory signals sᵢ(t) (i=1,2, . . . ).

9. A method in accordance with claim 8, wherein a consistency check takes place for the time indices kᵢ (i=1, 2, . . . ), in particular m, n and k, of the instantaneous respiratory rates.

10. A method in particular in accordance with claim 1, wherein a consistency check of the respiratory rates fᵢ(n) (i=1,2, . . . ) is carried out.

11. A method in accordance with claim 10, wherein the consistency check takes place by a comparison of the respiratory rates fᵢ(n) (i=1,2, . . . ) among one another.

12. A method in accordance with claim 11, wherein the differences between the respective respiratory rates fᵢ(n) (i=1,2, . . . ) is determined and is compared with a permitted tolerance A.

13. A method in accordance with claim 10, wherein only those respiratory rates are used for the weighted averaging of the respiratory rates fᵢ(n) (i=1,2, . . . ) which pass the consistency check.

14. A method in accordance with claim 1, wherein the signal quality is in particular determined via a consistency check and is optionally displayed.

15. A method, in particular in accordance with claim 1, comprising
the generation of at least two frequency signals FTᵢ(f) (i=1,2, . . . ) by transformation of at least two time dependent respiratory signals sᵢ(t) (i=1, 2, . . . ) into the frequency space
determination of a frequency signal FT(f) by a combination of the frequency signals FTᵢ(f) (i=1,2, . . . ),
wherein a respiratory rate f is determined on the basis of the frequency signal FT(f).

16. A method in accordance with claim 15, wherein the frequency signal FT(f) is determined by an averaging of the frequency signals FTᵢ(f) (i=1,2, . . . ).

17. A method in accordance with claim 15, wherein the respiratory rate f is determined by peak detection of the frequency signal FT(f).

18. A method in accordance with claim 15, wherein the respiratory rate f is determined by back transformation of the frequency signal FT(f)and an evaluation of the resulting signal s(t).

19. A method in accordance with claim 1, wherein the respiratory rate f is used for the initialization of the weighted averaging.

20. A method in accordance with claim 1, wherein the at least two time dependent respiratory signals sᵢ(t) (i=1,2, . . . ) are acquired from a PPG signal and an ECG signal.

21. A method in accordance with claim 1, wherein the at least two time dependent respiratory signals sᵢ(t) (i=1, 2, . . . ) form a selection from the following signals:
a respiratory signal determined from the heart rate s_{HR}(t),
a respiratory signal determined from the PPG signal s_{PPG}(t),
a respiratory signal determined from the PTT signal s_{PTT}(t),
a respiratory signal determined from the kurtosis of the ECG signal sₖᵤᵣₜ(t).

22. A method in accordance with claim 21, wherein at least three respiratory signals are used.

23. An apparatus for determining the respiratory rate of a patient by means of a method in accordance with claim 1.

24. An apparatus, in particular in accordance with claim 23, comprising a separate sensor unit for measuring the physiological signals from which the at least two time dependent respiratory signals can be determined and a processing unit for the evaluation of the data transmitted by the sensor unit.

25. An apparatus in accordance with claim 24, wherein the data generated by the sensor unit are transmitted to the processing unit in a wireless manner.

26. An apparatus in accordance with claim 24, wherein the sensor unit is fastened to the patient's wrist.

27. An apparatus in accordance with claim 24, wherein the at least two time dependent respiratory signals are determined from the physiological signals in the sensor units and are thereupon transmitted to the processing unit.

28. An apparatus in accordance with claim 23, comprising sensors for the measurement of the ECG signal and the PPG signal.

29. An apparatus in accordance with claim 28, wherein the heart rate, the pulse amplitude and the pulse wave transit time are determined from the ECG signal and the PPG signal.

30. An apparatus in accordance with claim 24, wherein the processing unit is part of a medical device, in particular of a medical device for extracorporeal blood treatment.

31. An apparatus in accordance with claim 24, wherein the processing unit is part of a computer network.
